(19) United States
(12) Patent Application Publication
(10) Pub. No.: US 2011/0277333 A1
(43) Pub. Date: Nov. 17, 2011

(54) HAIR DRYER CONTAINING A SILICONE HAIR CONDITIONING COATING

(75) Inventors: David W. Everett, Verona, WI (US); Richard A. Harthun, Eagle, WI (US); Maciej Murzynski, Verona, WI (US); Daniele Rosati, Verona, WI (US)

(73) Assignee: ROVCAL, INC., Madison, WI (US)

(21) Appl. No.: 13/099,975

(22) Filed: May 3, 2011

Related U.S. Application Data
(60) Provisional application No. 61/331,038, filed on May 4, 2010.

(51) Int. Cl. A45D 20/12; A61Q 5/12; A61K 8/89

(52) U.S. Cl. 34/60; 424/70.12; 132/221

(57) ABSTRACT

The present disclosure generally relates to a hair dryer comprising a protective grill having a composition deposited thereon, or present therein, for conditioning hair. More particularly, the conditioning composition comprises a cross-linked siloxane polymer and a silicone conditioning agent. Operation of the hair dryer initiates emission and transfer of the conditioning agent from the protective grill onto the hair surface of a user thereof.

### CROSS-REFERENCE TO RELATED APPLICATION

This application claims priority from U.S. Provisional Patent Application Ser. No. 61/331,038, filed May 4, 2010, the entire contents of which are incorporated herein by reference.

### FIELD OF THE DISCLOSURE

The present disclosure generally relates to a hair dryer comprising a protective grill having a composition deposited thereon, or present therein, for conditioning hair. More particularly, the conditioning composition comprises a cross-linked siloxane polymer and a silicone conditioning agent. Operation of the hair dryer initiates emission and transfer of the conditioning agent from the protective grill onto the hair surface of a user thereof.

### BACKGROUND OF THE DISCLOSURE

Hair dryers are personal grooming devices that blow air (either hot or cold) over wet or damp hair, in order to accelerate the evaporation of water particles therefrom and dry the hair. Typically, however, heat from the drying process damages various characteristics of hair, including, for example, the softness and overall condition of the hair.

Conditioning agents are commonly employed to enhance the manageability, health and/or appearance of the hair surfaces. A variety of conditioning agents are known, including various modified proteins and other film-forming natural and synthetic polymers, as well as silicones. Typically, these conditioning agents are in the form of a paste, cream, liquid, or gel, and are used by applying the agent directly to the hair surface.

Alternatively, however, conditioning agents, such as silicones, may be used by incorporating them into a hair care device. For example, the agents may be added to liquid coatings, applied to a surface of a hair care device and dried or cured to create a coating thereon. The resulting coatings may then transfer the conditioning agent (e.g., the silicone material) to hair surfaces that come into direct, physical contact therewith (i.e., the hair surface actually touches the surface of the hair care device).

Alternatively, the conditioning agent may be added to plastics used to manufacture the hair care devices, to provide beneficial conditioning and moisturizing properties. However, again, the agent is transferred from the plastic surface to hair through direct, physical contact with the hair care device.

While previously known methods for conditioning the hair by means of a hair care device may successfully impart some benefits to the hair, there are drawbacks. For example, the compositions and mixtures containing the conditioning agent are typically homogeneous or miscible. As such, it is difficult for the conditioning agent to separate from the mixture to provide maximum conditioning properties to the contact surface. More specifically, when a conventional silicone-containing composition is applied to the contact surface of a hair care device, the surface generally absorbs the entire composition, rather than just the conditioning agent. As such, repeated use of these compositions can lead to a build-up of undesirable effects, including, for example, a heavy, oily feel to the contact surface. Additionally, or alternatively, only a small fraction of the conditioning agent from the composition or mixture that is used transfers to the contact surface, which limits the life-time of the device (and/or increases the overall costs of the device).

Accordingly, there remains a need in the art for a hair care device, and more specifically a hair dryer, that can impart benefit to the condition of the hair while maximizing the useful life of the device.

### SUMMARY OF THE DISCLOSURE

Briefly, therefore, the present disclosure is directed to a hair dryer comprising a component having deposited thereon a composition for conditioning hair. The conditioning composition comprises a cross-linked siloxane polymer and a silicone conditioning agent. In one particular embodiment, the conditioning composition is in the form of a layer deposited on the surface of a protective grill, which is located at an opening (or an exhaust port) of the hair dryer through which heated air passes (or escapes) when in operation.

The present disclosure is also directed to a method for conditioning hair. The method comprises applying heated air to the hair using a hair dryer comprising a component over which the heated air passes, the component comprising a conditioning composition comprising a cross-linked siloxane polymer and a silicone conditioning agent, wherein the combination of the air temperature and the air velocity is sufficient to transfer the silicone conditioning agent from the conditioning composition to the hair surface. In one particular embodiment, the conditioning composition is in the form of a layer deposited on the surface of a protective grill, which is located at an opening (or an exhaust port) of the hair dryer through which heated air passes (or escapes) when in operation.

### BRIEF DESCRIPTION OF THE DRAWINGS

FIG. 1 depicts an exemplary hair dryer of an embodiment of the present disclosure.

FIG. 2 depicts an exemplary protective grill of a hair dryer of an embodiment of the present disclosure.

FIGS. 3A and 3B depict FTIR spectra, as further detailed in Example 1. In particular, FIG. 3A is a reference FTIR spectrum, illustrating the spectrum of the conditioning agent used, and FIG. 3B illustrates the FTIR spectrum obtained during analysis of a hair dryer comprising a protective grill prepared in accordance with the present disclosure.

It is to be noted that corresponding reference characters indicate corresponding parts throughout the several views of the drawings.

It is to be further noted that the design or configuration of the components presented in these figures are not to scale, and/or are intended for purposes of illustration only. Accordingly, the design or configuration of the components may be other than herein described without departing from the intended scope of the present disclosure. These figures should therefore not be viewed in a limiting sense.

### DETAILED DESCRIPTION OF THE DISCLOSURE

In accordance with the present disclosure, an improved method for conditioning the hair has been discovered, which utilizes the heat and air flow from a hair dryer to transfer a conditioning agent from the hair dryer to the hair surface. Accordingly, in accordance with the present disclosure, an improved hair dryer has additionally been discovered, which includes a conditioning agent suitable for transfer to the hair surface when the dryer is in operation.

More particularly, it has been discovered that a hair dryer may be prepared which has a conditioning agent incorporated into a component thereof, such as for example a protective grill (positioned at or near the point or opening of the dryer at which the heated air exits or escapes the device, the opening optionally being referred to herein as an exhaust port). It has further been discovered that by proper selection of a conditioning agent and the material in which the conditioning agent is incorporated, the temperature of operation of the hair dryer, and/or the air velocity of operation of the hair dryer, an amount of the conditioning agent may be transferred to the hair from the hair dryer component (e.g., the protective grill) upon which the conditioning agent is deposited (or in which the conditioning agent is incorporated).

Without being held to any particular theory, it is generally believed that when the hair dryer is activated, the air velocity causes the conditioning agent to be transferred from, for example, the component (e.g., the protective grill) surface to the hair surface (the conditioning agent becomes entrained in the air current or flow and is carried from the component surface to the hair surface). Additionally, and again without being held to any particular theory, it is generally believed that the elevated temperature of the air passing over the component (e.g., the protective grill) causes: (i) the conditioning agent present in the protective grill to migrate to the grill surface (either the grill itself, or a layer present on the grill surface thereof, as further detailed elsewhere herein); and/or (ii) the conditioning agent present on the protective grill surface to volatilize or vaporize, thus aiding the transfer thereof to the hair surface.

A. Terms

It is to be noted that as used herein, the following terms or phrases, or variations thereof, generally have the following meanings.

The term “hair dryer” generally refers to a device, such as a personal care device, used to dry wet or dampened hair. The term “hair dryer” also refers to a “hairdryer” or “blowdryer”. The term “hair dryer” also generally refers to a device used to style or fix hair into a desired configuration.

The term “protective grill” generally refers to a component of a hair dryer that is typically located in the exhaust of a hair dryer. The grill is typically affixed within the exhaust end of a hair dryer.

The term “cross-link” generally refers to chemical bonds that link one polymer chain to another.

The term “contact surface” generally refers to either the “subject contact surface”, or the surface of the subject that comes into contact with the device of the present disclosure. For example, “subject contact surface” can refer to the hair of the user of the device. Alternatively, “contact surface” may refer to the “device contact surface”, or the surface of the device that comes into contact with the surface of the subject.

The term “thermally stable” generally refers to a polymeric material that exhibits little or no chemical or compositional change when subjected to the operating temperatures of the hair dryers of the present disclosure (e.g., little or no breaks occur in the polymer backbone, or cross-links between polymer backbones). Additionally, or alternatively, this term generally refers to a polymeric material that exhibits little or no physical change (e.g., a change in hardness or brittleness) when subjected to the operating temperatures of the hair dryers of the present disclosure.

B. Conditioning Composition and Method of Preparation

In one embodiment of the present disclosure, the composition for conditioning hair that is deposited on the hair dryer component, such as the protective grill (as further detailed herein below), comprises, or consists essentially of, a cross-linked siloxane polymer and a silicone conditioning agent, and more particularly a thermally-stable, cross-linked siloxane polymer and a silicone conditioning agent. More specifically, and as further detailed below, the conditioning composition of the present disclosure comprises, or consists essentially of: (i) a cross-linked polydimethylsiloxane (PDMS) polymer that serves as a matrix or substrate, and (ii) a free or non-cross-linked PDMS conditioning agent, which is present (e.g., dispersed) in the cross-linked PDMS polymer (the free PDMS conditioning agent being able to move or migrate through the cross-linked PDMS polymer matrix or substrate, to the surface thereof, for transfer to the hair).

1. Preparation of Conditioning Composition

The cross-linked siloxane polymer is prepared from a mixture comprising, or consisting essentially of, two components. The first component thereof comprises, or consists essentially of, a high molecular weight siloxane polymer, such as a high molecular weight PDMS and a cross-linking agent. The high molecular weight silicone conditioning agent is typically present in the first component in an amount of from about 70% to about 90%, or, preferably from about 75% to about 85%, by weight of the first component. The cross-linking agent is typically present in the first component in an amount of less than about 0.2%, about 0.1%, or even about 0.05%, by weight of the first component. In this regard, it is to be noted that the concentrations may be other than herein described without departing from the scope of the present disclosure, and therefore should not be viewed in a limiting sense. For example, in particular embodiments of the present disclosure where the concentrations of the high molecular weight silicone conditioning agent and the cross-linking agent do not total 100% of the composition, various fillers and additives as further described herein may be present in the composition.

As previously noted, in one particular embodiment of the present disclosure, the high molecular weight silicone conditioning agent is high molecular weight polydimethylsiloxane. In this or another particular embodiment of the present disclosure, the cross-linking agent is a platinum-siloxane curing complex.

In this regard, it is to be noted that, as used herein, “high molecular weight” refers to a siloxane polymer, and more specifically PDMS, having a viscosity of greater than about 500 centistokes. In particular embodiments of the present disclosure, the high molecular weight siloxane polymer has a viscosity of about 600 centistokes, about 700 centistokes, about 800 centistokes, about 900 centistokes, or about 1,000 centistokes.

The second component of the mixture from which the cross-linked siloxane polymer is prepared comprises, or consists essentially of, a low molecular weight siloxane polymer, such as a low molecular weight PDMS. The low molecular weight silicone conditioning agent is typically present in the second component in an amount of from about 75% to about 95%, or about 80% to about 90%, by weight of the second component. In this regard, it is to be noted that the concentrations may be other than herein described without departing from the scope of the present disclosure, and therefore should not be viewed in a limiting sense. For example, in particular embodiments of the present disclosure where the concentration of the low molecular weight silicone conditioning agent does not total 100% of the composition, various fillers and additives as further described herein may be present in the composition.

As previously noted, in one particular embodiment of the present disclosure, the low molecular weight silicone conditioning agent is a low molecular weight polydimethylsiloxane.

In this regard, it is to be noted that, as used herein, “low molecular weight” refers to a siloxane polymer, and more specifically PDMS, having a viscosity of less than about 500 centistokes, about 350 centistokes, or even about 150 centistokes, the viscosity, for example, being in the range of between about 50 and less than about 500 centistokes.

Prior to combining the first and the second components, the contents of each component are mixed, and more specifically are typically mixed separately. That is, the contents of the first component are mixed together, the contents of the second component are mixed together, and then the mixture of the first component is combined with the mixture of the second component (or vice versa). Typically, the first component and the second component are mixed at a ratio of about 1:1 (weight), but in some instances may be mixed at a ratio, with either component in excess, of plus or minus about 5%, about 4%, about 3%, about 2% or about 1% of each component. For example, in one particular embodiment of the present disclosure, the first component and the second component are mixed at a ratio of about 1.05:1.0. In yet another particular embodiment of the present disclosure, the first component and the second component are mixed at a ratio of about 1.0:1.05. In this regard, other ratios may be used in various embodiments of the present disclosure without departing from the scope of the present disclosure and the recited ratios should not be viewed in a limiting sense.

In this regard, in a preferred embodiment of the present disclosure, the amounts of the second component (i.e., the “low molecular weight” component) and/or the first component (i.e., the “high molecular weight” component) that are combined or mixed, and thus the ratio thereof, in order to ultimately form the conditioning composition (as further detailed herein below), will in general be sufficient to ensure that the desired result is achieved. More specifically, the amount of the first and second components (or the ratio thereof) will in general be sufficient to ensure that the resulting conditioning component, or layer thereof, on the hair dryer (or more specifically the hair dryer component) contains (i) a sufficient quantity of free or non-cross-linked conditioning agent (e.g., free PDMS, either low molecular weight, high molecular weight, or a mixture thereof) to migrate to the conditioning composition layer (or more particularly the surface of the matrix) for transfer to the hair when the hair dryer is in use, and/or (ii) a sufficient quantity of cross-linked siloxane polymeric material so that the cross-linked siloxane polymeric material (and more specifically the matrix thereof) will continue to adhere to the surface of the hair dryer to which it is applied (e.g., the protective grill thereof).

Generally speaking, upon mixing the high molecular weight siloxane polymer and the cross-linking agent, and optionally additional filler or additives as described herein, of the first component (i.e., the high molecular weight component) the cross-linking begins. The cross-linking reaction being carried out by methods, and under conditions, generally known in the art; for example, the cross-linking may be allowed to proceed at about 80° C., 90° C., or even about 100° C., or more, for about 10 minutes, about 15 minutes, about 20 minutes or more. This reaction may be allowed to continue for a period of time to ensure the desired degree of cross-linking occurs (e.g., at least about 10%, 25%, 50%, 75%, or even about 100%) of the high molecular weight siloxane polymer, prior to addition of the second component (i.e., the low molecular weight component). For example, in one particular embodiment, about 100% cross-linking is achieved after about 20 minutes at about 100° C. Optionally, the second component may be added to the first component (or vice versa) while cross-linking is still occurring in the first component, such that a portion of the low molecular weight siloxane polymer is also cross-linked (e.g. about 5% or 10% and less than about 90%, 75%, 50%, or even about 25%).

In this regard, it is to be noted that cross-linking in the first component may be quenched, using methods generally known in the art, prior to addition of the second component thereto (or addition of the first component to the second component). Additionally, or alternatively, cross-linking may be quenched after the first component and the second component are combined. In this regard, it is to be further noted, generally speaking, cross-linking will be controlled to ensure that the resulting conditioning component, or layer thereof, on the hair dryer (or more specifically the hair dryer component) contains (i) a sufficient quantity of free or non-cross-linked conditioning agent (e.g., free PDMS, either low molecular weight, high molecular weight, or a mixture thereof) to migrate to the conditioning composition layer (or more particularly the surface of the matrix) for transfer to the hair when the hair dryer is in use, and/or (ii) a sufficient quantity of cross-linked siloxane polymeric material so that the cross-linked siloxane polymeric material (and more specifically the matrix thereof) will continue to adhere to the surface of the hair dryer to which it is applied (e.g., the protective grill thereof).

One or both of the components detailed above may optionally include additional additives that are suitable for use in the hair care industry. Suitable additives that can be added to one or both of the components include, but are not limited to, coloring agents (e.g., for aesthetics), sparkle additives (e.g., for aesthetics), and natural pearl additives (e.g., for conditioning). When added to one of the component mixtures, the coloring agent can be added in an amount from about 0.1% to about 0.3% by weight of the component. The sparkle additives can be added in an amount from about 0.5% to about 4% by weight of the component. The natural pearl additives can be added to the composition in an amount from about 0.1% to about 0.3% by weight of the component.

2. Resulting Layer of Conditioning Composition

In one embodiment of the present disclosure, a hair dryer is disclosed that is suitable for imparting a benefit to hair, such as an improved sensory feel. The hair dryer includes a component, such as a protective grill, which comprises a composition for conditioning hair. The conditioning composition can be applied to the hair dyer component, such as a protective grill, by various means known in the art, such as, for example, dipping the hair dryer component (e.g., protective grill) into a vat containing the conditioning composition, spraying the conditioning composition onto the component, or using a drop-wise means known in the art to apply the conditioning composition to the component.

The component, such as the protective grill, can be made of essentially any material generally known in the art, provided the material is compatible with the conditioning agent (or more generally the conditioning composition or coating that contains the conditioning agent present therein) and does not affect the transfer of the conditioning agent from the component to the user's hair. Additionally, the component material is preferably selected to ensure that the layer of the composition applied thereto remains adequately bound or adhered thereto for a sufficient period of time (e.g., the layer remains uniformly bound thereto for at least about 6 months, 12 months, 18 months, 24 months, etc.). In one exemplary embodiment, the component is made of a metal or a polymer-based material that provides adequate adhesion for the composition; for example, the component may be made of steel protected with a coat of paint.

The composition can be placed on any suitable surface or portion of the component. For example, in one particular embodiment of the present disclosure, the composition is placed on the back side of the protective grill (i.e., the side of the protective grill not facing the user).

In this regard, it is to be noted that, once applied, the conditioning composition may be subjected to any additional processes steps generally known in the art to ensure it is ready for use. For example, once applied to the component surface, the component may be allowed to stand (at, for example, ambient temperature and/or pressure, or alternatively at elevated temperature and/or pressure) to cure or set (e.g., evaporate solvent and/or dry) the coating or layer of the conditioning composition.

In this regard, it is to be further noted that, once applied (and optionally further processed to cure or set), the final conditioning composition may comprise, for example: (i) from about 5% to about 20%, or from about 10% to about 15%, by weight of the conditioning agent (e.g., the free, non-cross-linked low molecular weight PDMS); and/or (ii) from about 30% to about 40%, or about 35%, by weight of the conditioning agent (e.g., the free, non-cross-linked high molecular weight PDMS); and/or, (iii) from about 10% to about 40%, or about 20% to about 30%, by weight, of the cross-linked siloxane polymer. In this regard, it is to be noted that these concentrations may be other than herein described without departing from the scope of the present disclosure and the recited ranges should not be viewed in a limiting sense.

Although the amount of the conditioning composition on the hair dryer component may vary, typically the component contains between 1 and about 5 layers, or about 1 to about 3 layers, or about 1 layer, of the conditioning composition on the component. Additionally, or alternatively, the component, such as the protective grill, may comprise from about 0.01 grams to about 1.0 grams, or from about 0.1 grams to about 0.9 grams, or from about 0.25 grams to about 0.75 grams, of the conditioning composition thereon.

It should be noted that the cross-linked siloxane polymer typically has a viscosity that does not significantly affect (i) the application of the conditioning composition on the hair dryer component, and/or (ii) the ability of the free conditioning agent to migrate therefrom to the surface of the hair dryer component (e.g., the protective grill). For example, in one particular embodiment, the viscosity of the cross-linked siloxane polymer may be at least about 500 centipoise, and in some embodiments may be between about 400 and about 800 centipoise.

C. Exemplary Embodiments

As previously noted, the composition comprising the cross-linked siloxane polymer and the conditioning agent may be placed, for example, on the protective grill of a hair dryer. The following discussion is provided for purposes of illustration only, and, therefore, should not be viewed in a limiting sense.

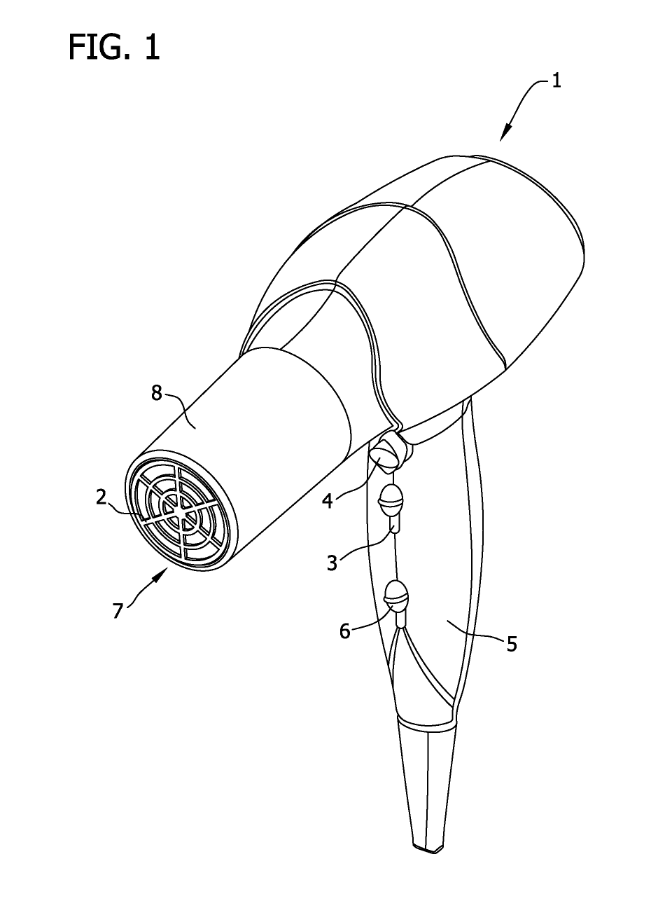

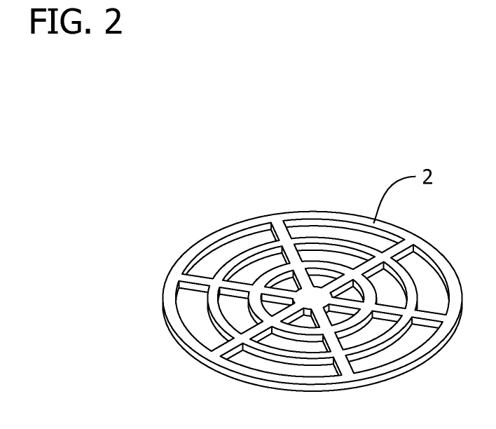

Referring to FIG. 1, an exemplary embodiment of a hair drying, in the context of a method for conditioning hair, will now be discussed (all in accordance with the present disclosure). A method for conditioning hair optionally comprises first coating a hair dryer component, such as a protective grill 2 of a hair dryer 1, positioned at or near an opening or exhaust port 7 (or point at which the air, which is typically heated, exits the hair dryer) of an exhaust tube 8 with a conditioning composition comprising a cross-linked siloxane polymer and a silicone conditioning agent. Referring now to FIG. 2, an exemplary protective grill 2 is shown. As noted above, the conditioning composition comprising the cross-linked siloxane polymer can be coated on any suitable part of the protective grill 2, including the side of the protective grill 2 that faces away from the user (i.e., the “back side” of the grill).

A method for conditioning the hair further comprises applying a combination of heat and air (or more specifically an air velocity), via the hair dryer 1 to the coating, wherein the combination of the heat and the air velocity transfers the free, non-cross-linked silicone conditioning agent from the coating on the protective grill 2 to a contact surface of a user of the hair dryer (not shown).

In accordance with an exemplary embodiment of the present disclosure, the hair dryer 1 comprises a handle 5. The handle 5 comprises an activating device 4 (e.g., an on/off button), a temperature control switch 3 and a velocity control switch 6. After the coating has been placed on an appropriate component (e.g., the protective grill 2), a user can activate the hair dryer 1 by pressing the activating device 4. Once the hair dryer 1 has been activated, the user can then regulate the temperature of the hair dryer 1 by adjusting the temperature control switch 3 and can regulate the air velocity passing through the exhaust tube 8 and exiting the exhaust port 7 by adjusting the velocity control switch 6. The hotter the temperature setting, the more readily the free, non-cross-linked silicone conditioning agent (e.g., the low molecular weight, high molecular weight, or combination thereof, conditioning agent that is free and non-cross-linked) will migrate to the surface of the cross-linked siloxane polymer matrix. Similarly, the faster the air velocity, the more readily the silicone conditioning agent will transfer from, in this embodiment, the protective grill 2 to the user's hair. After the heat has caused the low molecular weight silicone conditioning agent to separate from the high molecular weight silicone conditioning agent, the air velocity of the hair dryer 1 then transfers the low molecular weight silicone conditioning agent from the protective grill 2 to the user's hair (not shown).

It is to be noted that in all of the exemplary embodiments and disclosures discussed above, the present disclosure is able to be used on both humans and animals.

In this regard, it is to be noted that, over time, even at ambient temperatures, the free, non-cross-linked silicone conditioning agent (e.g., the low molecular weight silicone conditioning agent) may naturally migrate to the surface of the cross-linked siloxane polymer matrix. The application of heat to the cross-linked siloxane polymer matrix, however, accelerates the rate at which the conditioning agent (e.g., low molecular weight silicone conditioning agent) migrates to the surface of the cross-linked siloxane polymer matrix. Suitable temperatures that will accelerate the migration of the free, non-cross-linked silicone conditioning agent to the surface of the cross-linked siloxane polymer matrix are from at least about 100° C. to about 250° C., from about 125° C. to about 225° C., or from about 150° C. to about 200° C. When the hair dryer is operated at these temperatures, the free, non-cross-linked silicone conditioning agent (e.g., the low molecular weight silicone conditioning agent) separates (or more readily separates) from the cross-linked, siloxane polymer matrix and migrates to the surface of the protective grill.

Once the free, non-cross-linked silicone conditioning agent separates from the cross-linked, siloxane polymer matrix and migrates to the surface (of the hair dryer component, or the conditioning coating deposited therein), the hair dryer is then operated to apply a suitable air velocity (e.g., about 35, about 40, about 45, or even about 48 cubic feet per minute, or more) thereto. The air velocity causes or enables the free, non-cross-linked silicone conditioning agent to separate from the hair dryer component (e.g., the protective grill) surface, becoming airborne, and then being deposited on the surface of the hair of the user.

In this regard, it is to be noted that the distance over which the airborne conditioning agent may travel is at least in part dependent upon the air velocity exiting the exhaust port thereof; that is, as the air velocity increases, the distance over which the airborne conditioning agent will travel also typically increases. Accordingly, in one exemplary embodiment of the present disclosure, the hair dryer is operated at an air velocity of about 48 cubic feet per minute, and is placed about 1 inch from the surface of the hair of the user. In other particular embodiments of the present disclosure, the conditioning agent will travel about 2 inches, about 3 inches, about 4 inches or about 5 inches from the protective grill to the surface of the hair of the user.

In one embodiment of the present disclosure, the low molecular weight conditioning agent will continue to migrate from the protective grill after at least about 25 cumulative hours of usage of the hair dryer.

The following Example describes various embodiments of the present disclosure. Other embodiments within the scope of the appended claims will be apparent to a skilled artisan considering the specification or practice of the disclosure as described herein. It is intended that the specification, together with the Example, be considered exemplary only, with the scope and spirit of the disclosure being indicated by the claims, which follow the Example.

### EXAMPLE

The following non-illustrating example is provided to further illustrate the present disclosure.

### Example 1

### Release of Conditioning Agent onto a Contact Surface

The following example illustrates the release of a conditioning agent from a protective grill of a hair dryer onto a contact surface. As noted above, in an exemplary embodiment of the present disclosure, a protective grill of a hair dryer can be coated with a composition comprising a cross-linked siloxane polymer and a silicone conditioning agent, which can be in the form of a layer on the surface of the protective grill. During use and activation of the hair dryer, the combination of heat and air velocity causes the transfer of the silicone conditioning agent from the coating on the protective grill to a contact surface (e.g., hair).

The composition comprising a cross-linked siloxane polymer and a silicone conditioning agent was formed as discussed above and coated onto a protective grill of a hair dryer. The conditioning agent used in this example was polydimethylsiloxane.

The hair dryer was operated for about fifteen minutes and then turned off. After about fifteen minutes of rest, the hair dryer was then operated again for about fifteen minutes and then turned off again for about fifteen minutes. This cycle of about fifteen minutes of operation followed by about fifteen minutes of rest was continued for about 25 hours. After the about 25 hours of operation and rest, a metal coupon, free of any conditioning agent, was then placed in front of the hair dryer. The hair dryer was then activated and operated for about fifteen minutes. After about fifteen minutes of operation, the metal coupon was analyzed to determine whether or not the conditioning agent (i.e., the free, non-cross-linked conditioning agent) transferred from the coating on the protective grill to the metal coupon.

The transfer of the free, non-cross-linked conditioning agent from the coating on the protective grill to the metal coupon was evaluated via microscopic-Fourier transform infrared spectroscopy in the attenuated total reflectance (ATR) mode. Fourier transform infrared spectroscopy (FTIR) involves the study of molecular vibrations, wherein a continuous beam of electromagnetic radiation is passed through or reflected off the surface of a sample thereby causing individual molecular bonds and groups of bonds to vibrate at characteristic frequencies and absorb infrared radiation at corresponding frequencies. As a result, different molecules generate distinct patterns of absorption known as “spectra”, allowing one skilled in the art to characterize and identify certain molecules.

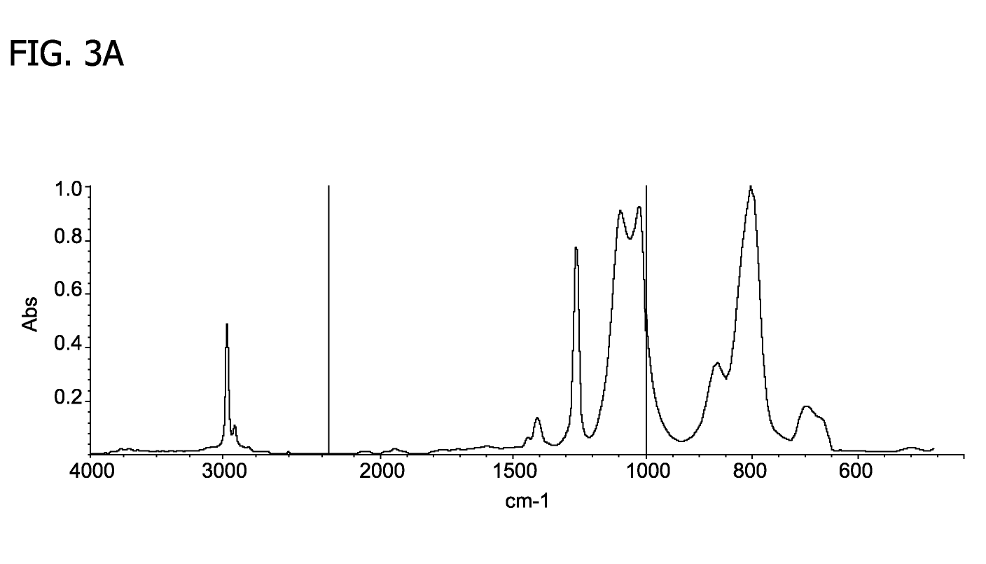

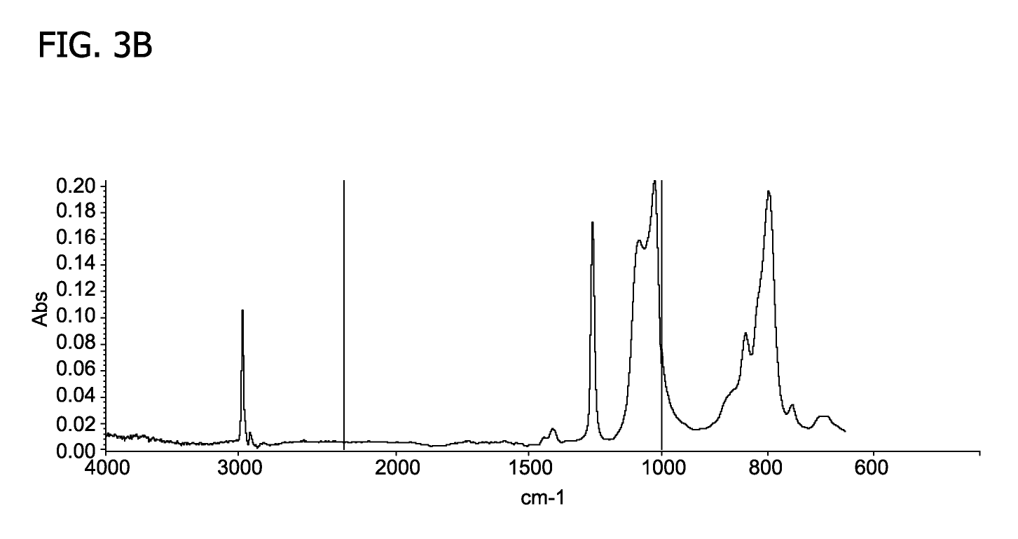

FIG. 3A provides the spectrum results of the reference sample conditioning agent (i.e., polydimethylsiloxane). As shown in FIG. 3A, the polydimethylsiloxane spectrum provides peaks at about 1300 cm⁻¹, 1100 cm⁻¹ and 800 cm⁻¹.

The reference metal coupon was directly analyzed following the about fifteen minutes of operation (that followed the about 25 hours of on again/off again hair dryer operation as detailed above), which produced the spectrum results in FIG. 3B. The spectrum results in FIG. 3B provide that the metal coupon has a presence of a conditioning agent at about 1300 cm⁻¹, 1100 cm⁻¹ and 800 cm⁻¹. Thus, the resulting spectrum in FIG. 3B confirmed the transfer of the free, non-cross-linked conditioning agent (i.e., the polydimethylsiloxane) from the coating on the protective grill to the contact surface (in this case, the metal coupon). Further, the spectrum in FIG. 3B also provides that after extensive use of the hair dryer, the free, non-cross-linked conditioning agent is not only still present on the protective grill but still continues to transfer from the grill to a user's hair.

In view of the above, it will be seen that the several advantages of the disclosure are achieved and other advantageous results attained. As various changes could be made in the above processes and composites without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements. The use of terms indicating a particular orientation (e.g., “top”, “bottom”, “side”, etc.) is for convenience of description and does not require any particular orientation of the item described.

What is claimed is:

1. A dryer for conditioning hair of a user, the hair dryer comprising a component having a hair conditioning composition deposited thereon, wherein the conditioning composition comprises a cross-linked siloxane polymer and a non-cross-linked silicone conditioning agent, and further wherein the conditioning component of the hair dryer is positioned within the hair dryer at or near an exhaust port through which heated air passes when the hair dryer is in operation.

2. The hair dryer of claim 1, wherein the conditioning component is a protective grill positioned at an exhaust port of an exhaust tube of the hair dryer.

3. The hair dryer of claim 2, wherein the cross-linked siloxane polymer forms a matrix in which the non-cross-linked silicone conditioning agent is dispersed and through which the non-cross-linked silicone conditioning agent may freely migrate in order to reach a surface thereof.

4. The hair dryer of claim 2, wherein the conditioning composition is in the form of a layer on a surface of the protective grill.

5. The hair dryer of claim 2, wherein the cross-linked siloxane polymer comprises a low molecular weight siloxane polymer and a high molecular weight siloxane polymer.

6. The hair dryer of claim 5, wherein the low molecular weight siloxane polymer and the high molecular weight siloxane polymer are both polydimethylsiloxanes having different average molecular weights.

7. The hair dryer of claim 1, wherein the conditioning composition comprises from about 10% to about 40% by weight of the cross-linked siloxane polymer.

8. The hair dryer of claim 1, wherein the non-cross-linked silicone conditioning agent is selected from the group consisting of a low molecular weight polydimethylsiloxane, a high molecular weight polydimethylsiloxane, and combinations thereof.

9. The hair dryer of claim 8, wherein the conditioning composition comprises from about 5% to about 20% by weight of the low molecular weight polydimethylsiloxane.

10. The hair dryer of claim 8, wherein the conditioning composition comprises from about 30% to about 40% by weight of the high molecular weight polydimethylsiloxane.

11. The hair dryer of claim 1, wherein the conditioning composition further comprises an additive selected from the group consisting of a coloring agent, a sparkle additive, a natural pearl additive and any combinations thereof.

12. A method for conditioning hair, comprising applying a velocity of heated air to the hair using a hair dryer comprising a component over which the heated air passes, the hair dryer component comprising a conditioning composition comprising a cross-linked siloxane polymer and a silicone conditioning agent, wherein the combination of the air temperature and the air velocity is sufficient to transfer the silicone conditioning agent from the conditioning composition to the hair surface.

13. The method of claim 12, wherein the conditioning component is a protective grill positioned at an exhaust port of an exhaust tube of the hair dryer.

14. The method of claim 13, wherein the cross-linked siloxane polymer forms a matrix in which the non-cross-linked silicone conditioning agent is dispersed and through which the non-cross-linked silicone conditioning agent may freely migrate in order to reach a surface thereof.

15. The method of claim 13, wherein the conditioning composition is in the form of a layer on a surface of the protective grill.

16. The method of claim 13, wherein the cross-linked siloxane polymer comprises a low molecular weight siloxane polymer and a high molecular weight siloxane polymer.

17. The method of claim 16, wherein the low molecular weight siloxane polymer and the high molecular weight siloxane polymer are both polydimethylsiloxanes having different average molecular weights.

18. The method of claim 12, wherein the conditioning composition comprises from about 10% to about 40% by weight of the cross-linked siloxane polymer.

19. The method of claim 12, wherein the non-cross-linked silicone conditioning agent is selected from the group consisting of a low molecular weight polydimethylsiloxane, a high molecular weight polydimethylsiloxane, and combinations thereof.

20. The method of claim 12, wherein the conditioning composition further comprises an additive selected from the group consisting of a coloring agent, a sparkle additive, a natural pearl additive and any combinations thereof.
